(19) United States
(12) Patent Application Publication
(10) Pub. No.: US 2003/0176871 A1
(43) Pub. Date: Sep. 18, 2003

(54) Device for the introduction of medical items

(76) Inventors: Paul Pavlov, Nijmwegen (NL); Claudio Bucci, Basel (CH); Martin Hess, Hoelstein (CH); Thomas Andreas Peter, Hoelstein (CH)

Correspondence Address: PENNIE & EDMONDS LLP, 1667 K STREET NW, SUITE 1000, WASHINGTON, DC, 20006

(21) Appl. No.: 10/360,620

(22) Filed: Feb. 10, 2003

Related U.S. Application Data
(63) Continuation of application No. PCT/CH01/00262, filed on Apr. 26, 2001.

(30) Foreign Application Priority Data
Aug. 10, 2000 (CH) CH 1566/00

(51) Int. Cl. A61F 11/00

(52) U.S. Cl. 606/108; 606/60

(57) ABSTRACT

The invention relates to a device for the introduction of medical items into the human or animal body. The device comprises a longitudinal guide sleeve or member having a first end segment, a second end segment, a longitudinal axis, and an outer surface. The second end segment defines a chamber and includes a drive element, which may be configured to couple to another drive device for rotatably driving the longitudinal member. The first end segment includes a screw thread for anchoring the device to bone such as a pedicle in the spine. The thread defines either a cylindrical or prismatic thread root or thread surface. The first end includes self-cutting teeth and at least one slot that extends along the outer surface between the self-cutting teeth and the second end segment in communication with the chamber.

### CROSS-REFERENCE TO RELATED APPLICATIONS

This application is a continuation of the U.S. national stage designation of copending International Patent Application PCT/CH01/00262, filed Apr. 26, 2001, which claims the benefit of application no. CH-1566/00 filed on Aug. 10, 2000 in Switzerland. The entire content of PCT/CH01/00262 is expressly incorporated herein by reference thereto.

### FIELD OF THE INVENTION

The present invention relates to a device for the introduction or insertion of medical items or instruments into the human or animal body.

### BACKGROUND OF THE INVENTION

In surgical operations, guides or protective sheaths are often used for the protection of bones or tissue, in particular the bone marrow and nerve roots, from potential injury caused by the surgical instruments employed. These guides or protective sheaths usually consist of a hollow profile or chamber, the interior geometry of which is configured in such a way that a medical instrument, or implant may be inserted and guided through the hollow profile and into, for example, the vertebral body and/or into the intervertebral disk area. Use of the protective sheath is particularly applicable when inserting medical instruments or implants that are expandable, i.e., may be enlarged in their total diameter upon exiting the front end of the sheath. A current problem with existing sheaths are that they are often insufficiently anchored to the bone into which the instrument or implant is to be inserted. Insufficient anchoring causes instability when radial, or axial stresses are exerted upon the sheath, thus providing the surgeon with an unstable guide path for instrument or implant insertion.

A trepan sheath is disclosed in European Patent No. EP-A-0 536 549 to Hiltebrandt. One disadvantage of this trepan sheath is that it is configured bluntly at its front end, and is therefore not suitable for anchoring into the bone tissue.

### SUMMARY OF THE INVENTION

The present invention is directed to a device for the insertion of medical instruments in a human or animal body comprising a longitudinal member having a longitudinal axis and an outer surface, a first end segment including at least a portion having a screw thread extending along the longitudinal axis for anchoring the longitudinal member, in which the screw thread defines a thread surface between the threads, and a second end segment for rotatably driving the longitudinal member, in which the second end defines a chamber. In addition, the first end segment includes self-cutting teeth for cutting into bone, the first end further includes at least one slot for distributing fluid implant material in a bone cavity and has at least a portion along the outer surface between the cutting teeth and the second end segment in communication with the chamber.

The invention is also directed to a device comprising a prismatic, or cylindrical guide sheath with a hollow space that essentially coaxially reaches through the guide sheath, and with drive means arranged at one end, which can be connected to drive devices, preferably a handle for inserting the device. A male thread is attached at the opposite end of the guide sheath, which has a cylindrical, or prismatic thread surface at a length L, and which serves for the fixation of the guide sheath in a bone, such as a pedicle.

The end of the guide sheath that is equipped with a male thread is embodied as self drilling, and/or self-cutting. This feature preferably eliminates the necessity of using drills and thread cutters, which in turn reduces the duration of the surgery. Furthermore, the self-drilling, and/or self-cutting features preferably ensure a geometrically optimum adjustment to the bone tissue, thus achieving the best possible anchoring of the device.

In a preferred embodiment, the guide sheath, and a handle serving as the drive device are constructed as two parts, whereby the drive means are preferably embodied as a male square, male hexagon, or thread. An externally operated (air, electricity) device, such as in the shape of an electric drill, can also be used as the drive device. In another embodiment of the device according to the invention, the guide sheath, and the drive means are constructed as one piece, whereby the drive means are embodied, for instance, as a hand wheel.

In another embodiment of the device according to the invention, the male thread has a thread gradient A at the front end of the guide sheath, and the ratio V between the length L of the cylindrical, or respectively prismatic, thread surface and the thread gradient A, V=L/A is within a range of 0.5≦V≦0.875. The thread profile preferably has an apex angle between 45° and 135°. Additionally, the male thread may have thread peaks that are rounded in the shape of a circular arc, or which are embodied elliptically convex. Preferably, thread height of the male thread is C=3 mm.

In yet another embodiment of the device according to the invention, the male thread can be embodied in the shape of a barb. For example, this may be achieved by a respectively asymmetrical embodiment of the thread shape, and by attaching notches to the male thread that are parallel to the longitudinal axis, or spirally shaped.

The thread geometry is embodied in such a way that an optimum of tear strength and bending stability occurs at the lowest possible tissue stress, such as by pedicles. One or multiple pitch, symmetrical, or asymmetrical threads can be used in various embodiments of the device according to the invention.

The device according to the invention can also be utilized in combination with a second, third, fourth, and/or additional hollow profile so that, for instance, the removal of a vertebral body, or of a intervertebral disk can be performed through a hollow profile, and the filling with oesteocementum through another, or the rinsing of a previously created cavity is possible.

In addition to instruments and implants, preparations, such as thermal, vibratorically effective, or optical devices can also be inserted through the guide sheath. The geometry of the guide sheath can preferably be determined by means of an exterior diameter of 3 to 15 mm, an interior diameter of 1 to 14 mm, and a length of 50 to 300 mm. Furthermore, the guide sheath can also be embodied as a bent, or bendable hollow profile.

In other embodiments of the device according to the invention, the first end of the guide sheath can be embodied with laterally attached holes, and/or completely, or partially closed in the front.

At least one advantage of the invention is that the geometry of the thread is such that low, and homogeneously distributed tension is induced in the bone under bending, pulling, or shearing stresses of the hollow profile, such as they occur in long surgical instruments, as they are used, for instance, in spine surgery. Additionally, the quality of anchoring in regard to bending stability and tear strength is optimized by means of the thread's geometry, and the self-drilling, and/or self-cutting characteristics of the Trepan end.

### BRIEF DESCRIPTION OF THE DRAWINGS

Preferred features of the present invention are disclosed in the accompanying drawings, wherein similar reference characters denote similar elements throughout the several views, and wherein:

FIG. 1 is a perspective view of an embodiment of the device according to the present invention;

FIG. 2 is a perspective view of another embodiment of the device according to the present invention;

FIG. 3 is a perspective view of the first end segment of the embodiment of the device according to the present invention;

FIG. 4 is a cross-sectional view of the screw thread at the first end segment of an embodiment of the device according to the present invention;

FIG. 5 is a perspective partial view of an additional embodiment of the device according to the present invention; and

FIG. 6 is a perspective partial view, of another embodiment of the device according to the present invention.

### DETAILED DESCRIPTION OF THE PREFERRED EMBODIMENTS

For convenience, the same or equivalent elements in the various embodiments of the invention illustrated in the drawings have been identified with the same reference numerals. Further, in the description that follows, any reference to either orientation or direction is intended primarily for the convenience of description and is not intended in any way to limit the scope of the present invention thereto.

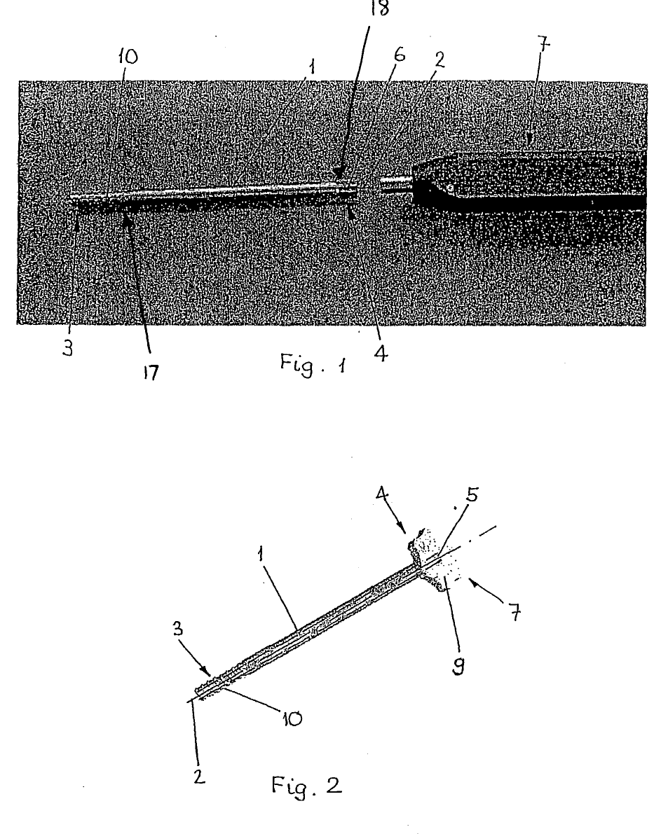

FIG. 1, shows a preferred embodiment of the device according to the present invention. The device comprises a cylindrical guide sheath or longitudinal member 1, having a longitudinal axis 2, a front sheath or first end segment 17, a rear sheath or second end segment 18, and a hollow body or chamber 5. In a preferred embodiment, hollow body 5 substantially coaxially penetrates through the guide sheath 1. Hollow body 5 is configured to receive medical instruments or implants. Alternatively, the hollow body may be configured to receive surgical preparations, such as thermal, vibratorically effective, or optical devices. In another embodiment of the device according to the invention, the first end segment 17 of the guide sheath may include lateral holes, which will be discussed in greater detail below. Referring to FIG. 4, the first end segment 17 may alternatively be completely, or partially closed at the face 11 of first end segment 17.

Referring again to FIG. 1, guide sheath 1 is shown as substantially straight. Alternatively, guide sheath 1 may include an arcuate portion or bend. Drive means or drive element 6 is configured as a hexagon, which may be coupled to a drive device 7 embodied as a handle. Alternatively drive element 6 may be configured as a square or a thread, and drive device 7 may be a pneumatic or electric device such as an electric drill. Drive element 6 and device 7 are arranged at the rear sheath segment 18, such that the guide sheath 1 can be hand-tightened into a bone, such as a pedicle in the spine. The first end segment 17 has a first end 3 that includes a male thread or screw thread 10. Referring to FIG. 4, thread 10 defines a thread surface 15 between the thread flanks having a length L for the fixation of the guide sheath 1 in a bone. The thread surface may have a cylindrical cross-section. Alternatively, the thread surface may have a prismatic cross-section.

The embodiment of the device according to the invention illustrated in FIG. 2 differs from that in FIG. 1 only in that the drive means 6, and the drive device 7 are constructed as one piece, whereby the drive device is embodied as a hand wheel 9.

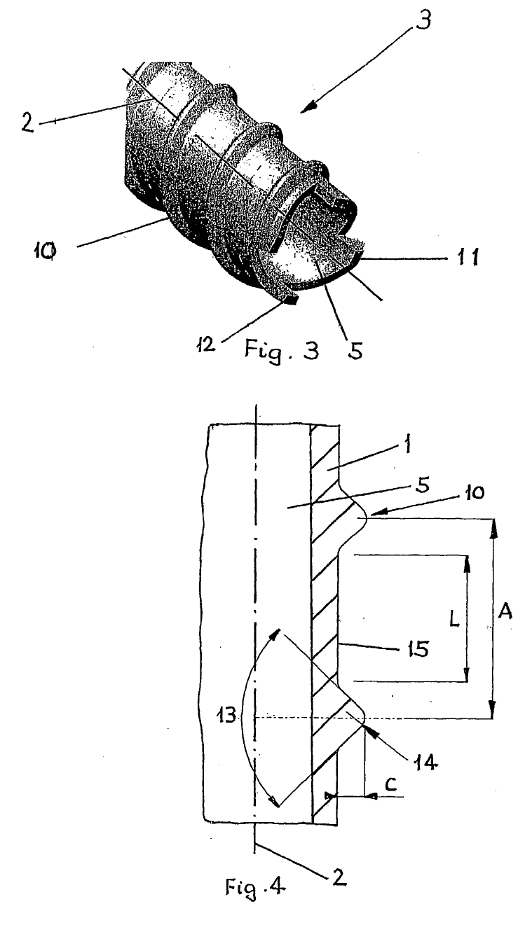

FIG. 3 shows a portion the first end 3 of first end segment 17 in one embodiment of the device according to the present invention. Cutting teeth 12 are arranged at the face 11 of the first end 3 of the guide sheath 1, which facilitate self-drilling. Depending on the embodiment of the cutting teeth 12, first end segment 17 may further include thread 10 that is self-cutting. Providing thread having this feature preferably eliminates the necessity of using drills and thread cutters, thereby reducing the duration of the surgery. Furthermore, the self-drilling and/or self cutting characteristics of the device facilitates a geometrically optimum adjustment to the bone tissue.

Referring back to FIG. 4, detailed features of male thread 10 are shown. Thread 10 includes a thread gradient A, in the case of a single thread, or in the case of double or multiple threads, the thread gradient is equivalent to a multiple of A. In a preferred embodiment, ratio V of the length L of the thread surface 15 and the thread gradient A, V=L/A, is within a range of 0.5≦V≦0.875. In another embodiment, the thread profile has an apex angle or thread angle 13 between 45° and 135°. In another embodiment, the male thread 10 is comprised of thread peaks 14, which are rounded in the shape of a circular arc. Alternatively, the thread peaks may be elliptically convex. In yet another embodiment, the thread 10 has a thread height C, and C≦3 mm. The thread 10 may be alternatively, advantageously provided with an asymmetrical thread profile. In yet another embodiment of the device according to the invention, the thread 10 may be formed as a barb. This formation may be achieved by providing an asymmetrical thread profile with notches in the thread 10, such that the notches are parallel to the longitudinal axis. Alternatively, the notches may be spirally shaped.

Referring now to FIG. 5, another embodiment of the device according to the invention is shown that differs from the embodiments according to FIGS. 1-4 in that the guide sheath 1 at the first end segment 17 has a plurality of radial perforations or slots 28 that are positioned along and penetrate the jacket or outer surface of the guide sheath I into the hollow space 5. The perforations 28 can be embodied as a plurality of elongated holes, the long axes of which are arranged parallel to the longitudinal axis 2 of the guide sheath 1. Alternatively, instead of an embodiment of the perforations 28 as elongated holes, round, or square perforations may also be provided.

By providing first end segment 17 with perforations 28, an improved distribution of pastes, solutions, suspensions or other fluid implant material can be achieved at the point of insertion.

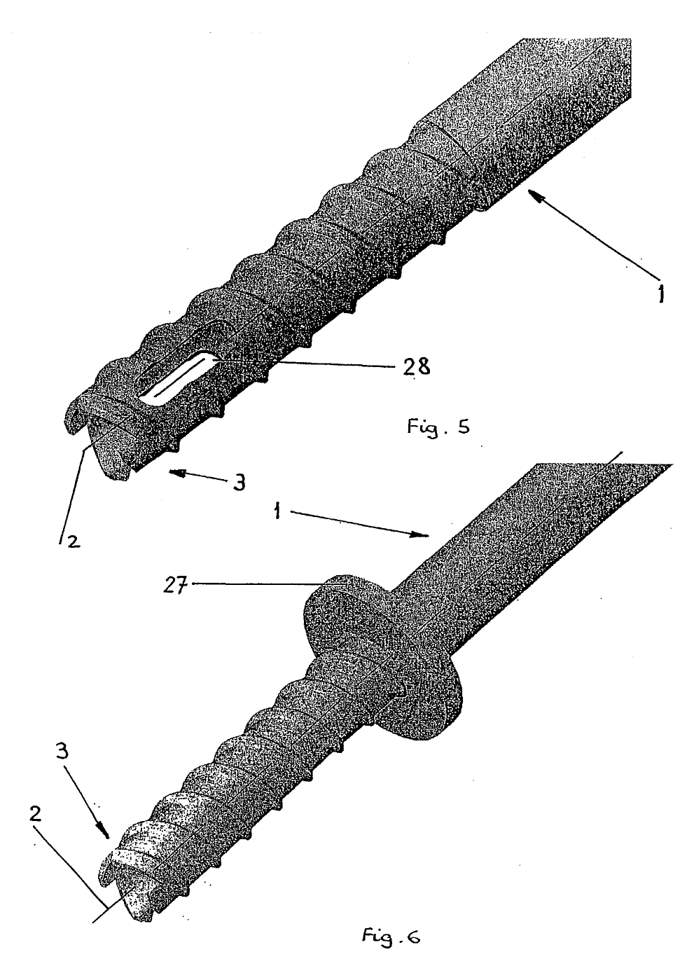

Another embodiment of the device according to the invention is shown in FIG. 6. This embodiment differs from those according to FIGS. 1-5 in that it is comprised of a stop unit 27. This stop unit 27 is configured as a circular ring concentric to the longitudinal axis 2 of the guide sheath 1 so that the guide sheath 1 can be screwed into a body up to a depth that is limited by the stop unit 27.

In another embodiment, the device according to the present invention may be utilized in combination with a second, third, fourth, and/or additional guide sheath positioned relative to the first guide sheath. Such a configuration may be advantageous when for instance, removing a vertebral body, or an intervertebral disk. The removal can be performed through one guide sheath, and the filling of the cavity with, for example, oesteocementum, may be performed using another guide sheath. Alternatively, the second guide sheath may be used to rinse a previously created cavity.

In a preferred embodiment, guide sheath 1 may be dimensioned such that it includes an exterior diameter of 3 to 15 mm, an interior diameter of 1 to 14 mm, and a length of 50 to 300 mm.

It will be appreciated that when using the device, sufficient anchoring of the guide sheath in the bone may be achieved. The device provides a guide sheath that is temporarily anchored, for example percutaneously to the spine, more specifically to the pedicle of the vertebrae, thereby providing minimally invasive, or open access to the spine. It will be further appreciated that the device may provide protection for the vertebral pedicle, for example, including its exterior bone marrow and nerve roots, preventing potential damage to the tissue due to inserted medical instruments and implants. In addition, the device may provide a pathway for guiding medical instruments, at the point of insertion in the body, such as curettes, and implants thereby providing translatory stability and limitations on the rotational movement of an inserted medical instrument or implant that expands upon exiting the front end of the guide sheath. It may further be appreciated that the device according to the present invention may provide an improved distribution of viscous or otherwise flowing implants such as bone filling material that is, for example, in the form of a paste, solution, or suspension.

It will be appreciated that, when using the device according to present invention, relatively low, and homogeneously distributed tensions are induced in the bone during surgical procedures in which the bone is anchored. The geometry of the thread is such that despite the bending, pulling, or shearing stresses experienced by the hollow body 5 of the guide sheath 1, due to the manipulation of inserted long surgical instruments during, for example, spine surgery, relatively low and homogeneously distributed tensions are induced in the bone. Additionally, the anchoring characteristics of the device, with respect to bending stability and tear strength, are optimized by both the geometry of the thread 10, and the self-drilling, and/or self-cutting characteristics of the device at its first end segment 17.

While various descriptions of the present invention are described above, it should be understood that the various features can be used singularly or in any combination thereof. Therefore, this invention is not to be limited to only the specifically preferred embodiments depicted herein. Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A device for the insertion of medical items in a human or animal body comprising:
a longitudinal member having a first end segment, a second end segment, a longitudinal axis, and an outer surface;
the first end segment including at least a portion having a screw thread extending along the longitudinal axis for anchoring the longitudinal member in bone, the screw thread defining a thread surface;
the second end segment configured for rotatably driving the longitudinal member, the second end segment defining a chamber; and
wherein the first end segment includes self-cutting teeth for cutting into bone, the first end further including at least one slot for distributing fluid implant material in a bone cavity, the slot having at least a portion along the outer surface between the cutting teeth and the second end segment in communication with the chamber.

2. The device of claim 1, wherein at least a portion of the longitudinal member is substantially arcuate.

3. The device of claim 1, wherein the chamber extends along the longitudinal axis from the first end segment to the second end segment substantially therethrough.

4. The device of claim 1, wherein the screw thread surface has a substantially cylindrical cross-section.

5. The device of claim 1, wherein the screw thread surface has a substantially prismatic cross-section.

6. The device of claim 1, wherein the screw thread has a thread gradient A and the thread surface is of length L along the longitudinal axis, wherein further the ratio V of length L to thread gradient A is 0.5≦V≦0.875.

7. The device of claim 6, wherein the screw thread is a multiple pitched thread.

8. The device of claim 1, wherein the screw thread is asymmetrical.

9. The device of claim 1, wherein the screw thread has a thread profile defining a thread angle between 45° and 135°.

10. The device of claim 1, wherein the screw thread is self-threading.

11. The device of claim 1, wherein the screw thread is formed as a barb.

12. The device of claim 1, wherein the screw thread has a thread profile defining thread peaks that are substantially round.

13. The device of claim 1, wherein the screw thread has a thread profile defining thread peaks that are elliptically convex.

14. The device of claim 1, wherein the second end segment includes a handle for manually rotatably driving the longitudinal member.

15. The device of claim 1, wherein the second end segment includes a drive element configured for coupling the longitudinal member to a drive device for rotatably driving the longitudinal member.

16. A device for the insertion of medical items in a human or animal body comprising:
a longitudinal member having a first end segment, a second end segment, and a longitudinal axis;
the first end segment including at least a portion having a screw thread extending along the longitudinal axis for anchoring the longitudinal member, the screw thread defining a thread surface;
the second end segment configured for rotatably driving the longitudinal member, the second end segment defining a chamber; and
wherein the screw thread has a thread gradient A and the thread surface is of length L along the longitudinal axis, wherein further the ratio V of length L to thread gradient A is 0.5≦V≦0.875.

17. The device of claim 16, wherein the screw thread is a multiple pitched thread.

18. The device of claim 16, wherein the longitudinal member has a stop element for limiting the depth that the device is anchored into bone.

19. A device for the insertion of medical items in a human or animal body comprising:
a first longitudinal member for inserting an instrument for drilling into a bone,
a second longitudinal member for inserting fluid implant material into the bone;
each first and second longitudinal member comprising:
a longitudinal axis and an outer surface;
a first end segment including at least a portion having a screw thread extending along the longitudinal axis for anchoring the longitudinal member, the screw thread defining a thread surface;
a second end segment configured for rotatably driving the longitudinal member, the second end segment defining a chamber; and
wherein the second longitudinal member is positioned relative to the first longitudinal member, and the first end segment of at least one of the first and second longitudinal members includes self-cutting teeth for cutting into bone.

20. The device of claim 19, wherein the chamber of at least one of the first and second longitudinal members extends along the longitudinal axis from the first end segment to the second end segment substantially therethrough.

21. The device of claim 19, wherein the first end segment of at least the second longitudinal member includes at least one slot for distributing fluid implant material in bone cavities, the slot having at least a portion along the outer surface between the first and second end segments in communication with the chamber.
